(19) United States
(12) Patent Application Publication
(10) Pub. No.: US 2002/0004584 A1
(43) Pub. Date: Jan. 10, 2002

(54) HIV immune adjuvant therapy

(76) Inventor: Mark A. Laughlin, Edison, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(21) Appl. No.: 09/801,980

(22) Filed: Mar. 8, 2001

Related U.S. Application Data
(60) Provisional application No. 60/188,338, filed on Mar. 9, 2000.

(51) Int. Cl. A61K 38/21

(52) U.S. Cl. 530/351; 424/85.7

(57) ABSTRACT

Methods for promoting an HIV-1 specific immune response in adult and pediatric patients having HIV-1 infections as well as patients co-infected with HIV-1 and HCV involving administering a therapeutically effective amount of pegylated interferon-alfa, e.g., pegylated interferon alfa-2b are disclosed.

### BACKGROUND OF THE INVENTION

The present invention relates to methods of promoting an immune response to immunodeficiency virus type-1 (“HIV-1 ”) in patents infected with HIV-1 by administering to such patients an effective amount of interferon-alfa.

A -M. Vandamme et al., Antiviral Chemistry & Chemotherapy, 9:187-203 (1998) disclose current clinical treatments of HIV-1 infections in man including at least triple drug combinations or so-called Highly Active Antiretroviral Therapy (“HAART”); HAART involves various combinations of nucleoside reverse transcriptase inhibitors (“NRTI”), non-nucleoside reverse transcriptase inhibitors (“NNRTI”) and HIV protease inhibitors (“PI”). In compliant patients, HAART is effective in reducing mortality and progression of HIV-1 to AIDS. However, these multidrug therapies do not eliminate HIV-1 and long-term treatment often results in multidrug resistance. Discontinuation of anti-retroviral therapy, e.g. HAART, in HIV-1 infected patients has resulted in a rapid increase in HIV-RNA plasma levels, in most patients. A concomitant increase in HIV-1 specific cytotoxic T-lymphcytes is often observed. The effectiveness of these increased T-cells in limiting HIV-1 replication , however, is in question, given the high viremia observed with anti retroviral therapy interruption. Cessation of HAART followed by daily doses of IL-2 has been reported to promote immunity to HIV-1 in some patients but HIV-1 viral load rebounded upon discontinuation of HAART with or without hydroxyurea.

Recent reports of preliminary results of structured treatment interruption (“STI”) in use of HAART in HIV-1 patients to induce an immune response to HIV-1 have been published (Structured Treatment Interruptions Workshop Summary published Jan. 31, 2000, pages 1-21). Maintenance of HIV-1 viral suppression during a STI is only observed in a minority of HIV-1 patients who discontinued HMRT. In other individuals who repeatedly discontinued HAART, during the discontinuation of HAART HIV-1 immune specific cells were restored during the STlof HAART, but they were rapidly destroyed by HIV-1 replication. Cessation of HAART followed by daily doses of IL-2 has been reported to promote immunity to HIV-1 in some patients but HIV-1 viral load rebounded upon discontinuation of HMRT with or without hydroxyurea.

Development of new drug therapies to provide an enhanced immune response to HIV-1 infections, especially during interruptions in HAART treatment remains a priority.

### SUMMARY OF THE INVENTION

The present invention provides a method of promoting an HIV-1 specific immune response in a patient having an HIV-1 infection in need of such promoting which comprises administering to such a patient an effective amount of interferon-alfa.

The present invention provides a method of promoting a HIV-1 specific immune response in a patient having an HIV-1 infection in need of such promoting which comprises administering to such a patient an effective amount of pegylated interferon alfa.

The present invention also provides a method of promoting HIV-1-specific T-cell activity in a patient having an HIV-1 infection who has discontinued anti-HIV therapy which comprises administering to such a patient an amount of interferon-alpha for a time sufficient to lower HIV-RNA plasma level of the patient to a level below the patient's HIV-RNA plasma level prior to initiation of the anti-HIV-therapy.

The present invention also provides a method of promoting HIV-1-specific T-cell activity in a patient having an HIV-1 infection who has discontinued anti-HIV therapy which comprises administering to such a patient an amount of pegylated interferon alpha for a time sufficient to lower HIV-RNA plasma level of the patient to a level below the patient's HIV-RNA plasma level prior to initiation of the anti-HIV-therapy.

The present invention also provides a method of promoting HIV-1-specific T-cell activity in a patient having an HIV-1 infection which comprises administering to such a patient an effective amount of interferon alpha in association with an effective amount an anti-HIV therapy for a time sufficient to effect such promoting.

The present invention also provides a method of promoting HIV-1-specific T-cell activity in a patient having an HIV-1 infection which comprises administering to such a patient an effective amount of pegylated interferon alpha in association with an effective amount an anti-HIV therapy for a time sufficient to effect such promoting.

### DETAILED DESCRIPTION

Discontinuation of anti-retroviral therapy, e.g. HAART, in HIV-1 infected patients normally results in a rapid increase in HIV-RNA plasma levels. One preferred embodiment of the present invention provides a method of administering pegylated interferon alpha to promote an HIV-specific immune response in HIV-1 infected patients who have discontinued an anti-retroviral therapy, especially HAART. The pegylated interferon alfa may be administered once weekly to promote an HIV-specific immune response, following discontinuation of HAART Twice weekly dosing of pegylated interferon alpha may be used if the HIV-RNA plasma levels continue to rise rapidly after discontinuation of HAART. Administration of pegylated interferon alfa is continued after cessation of HAART for a time sufficient to lower HIV-RNA plasma levels below the initial HIV-RNA plasma levels prior to initiation of HAART. Normally, 4-8 weeks of administering interferon-alfa, preferably pegylated interferon alfa is a sufficient period of time to achieve such a reduction but the precise dose and dose regimen will be determined by the treating clinician taking into consideration the initial HIV-RNA viral load, absolute or relative percent of CD 4 cells, the age and medical condition of the patient. The re-initiation of HAART is continued for a time sufficient to to lower HIV-RNA plasma levels below detectable limits, i.e. below 50 HIV-RNA copies per mL of plasma. The period of time is normally about a year,bu the exact period of time will be determined in accordance with good clinical practice to minimize HIV-1-RNA plasma levels. See for example A-M. Vandamme et al., in Antiviral Chemistry & Chemotherapy, 9:187-203 (1998) and “Drugs for HIV Infection” in The Medical Letter Vol. 39 (Issue 1015) Dec. 5, 1997, pages 111-116. Another embodiment of the present invention provides a method of administering pegylated interferon alfa to promote an HIV specific immune response when administered with HAART. The administration of pegylated interferon alfa with HAART would be for a sufficient time to achieve immune response to allow a STI resulting in a maintained viral suppression to a level below that prior to initiation of anti-retroviral therapy. In a more preferred embodiment of the present invention, there are at least three STI's wherein pegylated interferon alfa is administered to promote an HIV specific immune response Therafter HAART need not be re-initiated if the patient has less than about 10,000 HIV-RNA copies per ml of plasma, preferably less than about 5,000 HIV-RNA copies per ml of plasma.

The term “HIV-1-specific immune response” as used herein means any immune response which leads to decreased HIV-RNA plasma levels, including, but not limited to promoting HIV-1 specific T-cell activity, proliferation of T-cells such as cytotoxic T-lymphocytes and cytokines and chemokines, such as interleukins, e.g. IL-2 and interferon, e.g. interferon-gamma.

The term “HIV-1 specific cells” as used herein includes, but is not limited to, T-lymphocytes, e.g. CD4+-T-cells, CD8+-T-cells.

The terms “anti-retroviral therapy” and “anti-HIV-1 therapy” as used herein means the multi-drug therapies used in current clinical treatments of HIV-1 infections, including but not limited to the multi-drug anti-HIV-1 therapies, e.g., the triple and quadruple anti-HIV-1 drug therapies (HAART) such as disclosed by A-M. Vandamme et al., Antiviral Chemistry & Chemotherapy, 9:187-203 (1998) which describes the current clinical treatments of HIV-1 infections, including when to start multi-drug therapy and which drugs to combine. The triple drug therapy may include two nucleoside and nucleotide reverse transcriptase inhibitors (“NRTIs”) and one protease inhibitor (“PI”), but there are many issues to be considered in the choice of the precise HAART for any patient. See for example, Tables 1 & 2 and FIG. 2 in A-M. Vandamme et al., and “Drugs for HIV Infection”, listed hereinabove.

The term “a patient having an HIV-1 infection” as used herein means any patient -including a pediatric patient-having HIV-1 infection and includes treatment-naive patients and treatment-experienced patients having the HIV-1 infection as well as treatment-naive patients and treatment-experienced patients co-infected with the HIV-1 and hepatitis C virus (“HIV”).

The term “pediatric patient” as used herein means a patient below the age of 17, and normally includes those from birth to 16 years of age.

The term “treatment-naive patient” as used herein means any patient having HIV-1 or co-infected with the HIV-1 and HCV who have never been treated with any anti-retroviral drugs, e.g., NRTI, NNRTI, PI or any interferon, including but not limited to interferon-alfa, or pegylated interferon alfa.

The term “treatment-experienced patient” as used herein means any patient having HIV-1 or co-infected with the HIV-1 and HCV who have initiated some form of anti HIV therapy including, but not limited to HAART or some form of anti-HCV therapy, including but not limited to interferon-alfa, pegylated interferon alfa or ribavirin as well as those patients undergoing HAART who have undetectable HIV-RNA plasma levels.

The term “patients having hepatitis C infections” as used herein means any patient-including a pediatric patient-having hepatitis C and includes treatment-naive patients having hepatitis C infections and treatment-experienced patients having hepatitis C infections as well as those pediatric, treatment-naive and treatment-experienced patients having chronic hepatitis C infections.

These patients having hepatitis C include those who are infected with mutiple HCV genotypes including type 1 as well as those infected with,e.g., HCV genotypes 2, 3, 4, 5 and/or 6 and other possible HCV genotypes.

The term “treatment-naive patient having hepatitis C infections” as used herein means patient with hepatitis C who has never been treated with ribavirin or any interferon, including but not limited to interferon-alfa, or pegylated interferon alfa.

The term “treatment-experienced patients having hepatitis C infections” as used herein means patients with hepatitis C who have been treated with ribavirin or any interferon, including but not limited to interferon-alfa, or pegylated interferon alfa, including relapsers and non-responder.

The term “relapsers” as used herein means treatment-experienced patients with hepatitis C who have relapsed after initial response to previous treatment with interferon alone, or in combination with ribavirin.

The term “non-responders” as used herein means treatment-experienced patients with hepatitis C who have not responded to prior treatment with any interferon alone, or in combination with ribavirin.

When the pegylated interferon-alfa administered is a pegylated interferon alfa-2b, the therapeutically effective amount of pegylated interferon alfa-2b administered during the treatment in accordance with the present invention, including in first and second treatment time periods, is in the range of about 0.1 to 9.0 micrograms per kilogram of pegylated interferon alfa-2b administered per week, in single or divided doses, preferably once a week (QW) or twice a week(BIW), preferably in the range of about 0.1 to about 9.0 micrograms per kilogram of pegylated interferon alfa-2b administered once a week (QVV) or in the range of about 0.05 to about 4.5 micrograms per kilogram of pegylated interferon alfa-2b administered twice a week(BIW), or is in the range of about 0.5 to about 3.0 micrograms per kilogram of pegylated interferon alfa-2b administered per week, preferably in the range of about 0.5 to about 3.0 micrograms per kilogram of pegylated interferon alfa-2b administered once a week (QW) or in the range of about 0.25 to about 1.5 micrograms per kilogram of pegylated interferon alfa-2b administered twice a week , or is in the range of about 0.75 to about 1.5 micrograms per kilogram of pegylated interferon alfa-2b administered per week, most preferably is in the range of about 0.75 to about 1.5 micrograms per kilogram of pegylated interferon alfa-2b administered once a week or about 0.375 to about 0.75 micrograms per kilogram of pegylated interferon alfa-2b administered twice a week.

When the pegylated interferon-alfa administered to pediatric patients is a pegylated interferon alfa-2b, the therapeutically effective amount of pegylated interferon alfa-2b administered during the treatment in accordance with the present invention, including in first and second treatment time periods is in the range of about 0.1 to 9.0 micrograms per kilogram of pegylated interferon alfa-2b administered per week, in single or divided doses, preferably once a week (QW) or twice a week(BIW), more preferably about 0.1 to about 9.0 micrograms per kilogram of pegylated interferon alfa-2b administered once a week (QW), or about 0.05 to about 4.5 micrograms per kilogram of pegylated interferon alfa-2b administered per week, in single or divided doses, preferably once a week (QW) or twice a week(BIW), more preferably about 0.05 to about 4.5 micrograms per kilogram of pegylated interferon alfa-2b administered once a week, or preferably about 0.75 to about 3.0 micrograms per kilogram of pegylated interferon alfa-2b administered in single or divided doses, preferably once a week (QW) or twice a week(BIW), more preferably about 0.75 to about 3.0 micrograms per kilogram of pegylated interferon alfa-2b administered once a week or about 0.375 to about 1.5 micrograms per kilogram of pegylated interferon alfa-2b administered twice a week, and most preferably about 2.25 to about 2.6 micrograms per kilogram of pegylated interferon alfa-2b administered once a week or about 1.125 to about 1.3 micrograms per kilogram of pegylated interferon alfa-2b administered twice a week(BIW). In a preferred embodiment of the present invention, pediatric doses of about 0.75, about 1.5 and about 3.0 micrograms per kilogram of pegylated interferon alfa-2b are administered once a week

When the pegylated interferon-alfa administered is a pegylated interferon alfa-2a, the therapeutically effective amount of pegylated interferon alfa-2a administered during the treatment in accordance with the present invention, including in first and second treatment time periods, is in the range of about 50 micrograms to about 500 micrograms once a week(“QW”), preferably about 200 micrograms to about 250 micrograms QW or the effective amount is in the range of about 50 micrograms to about 250 micrograms twice a week, preferably about 100 micrograms to about 125 micrograms twice a week.

When the pegylated interferon-alfa administered to a pediatric patient is a pegylated interferon alfa-2a, the therapeutically effective amount of pegylated interferon alfa-2a administered during the treatment in accordance with the present invention, including in first treatment time period is in the range of about 50 micrograms to about 500 micrograms once a week(“QW”), preferably about 300 micrograms to about 375 micrograms QW or the therapeutically effective amount of pegylated interferon alfa-2a administered to a pediatric patient is in the range of about 50 micrograms to about 250 micrograms twice a week, preferably about 150 micrograms to about 190 micrograms once a week

Ribavirin is administered to the patient in association with pegylated interferon-alfa, that is, before, after or concurrently with the administration of the pegylated interferon alfa. The pegylated interferon-alfa dose is preferably administered during the same period of time that the patient receives doses of ribavirin. The amount of ribavirin administered concurrently with the pegylated interferon-alfa is from about 400 to about 1600 mg per day, preferrably about 600 to about 1200 mg/day or about 800 to about 1200 mg day and most preferably about 1000 to about 1200 mg/kg a day. The pegylated interferon-alfa dose is also preferably administered to the pediatric patient during the same period of time that such patient receives doses of ribavirin. The amount of ribavirin administered to the pediatric patient concurrently with the pegylated interferon-alfa is from about 8 to about 15 mg per kilogram per day, preferrably about 8, 12 or 15 mg per kilogram per day, in divided doses.

Pegylated interferon-alfa formulations are not effective when administered orally, so the preferred method of administering the pegylated interferon-alfa is parenterally, preferably by subcutaneous, IV, or IM, injection. Ribavirin may be administered orally in capsule, tablet or liquid form in association with the parenteral administration of pegylated interferon-alfa . Of course, other types of administration of both medicaments, as they become available are contemplated, such as by nasal spray, transdermally, by suppository, by sustained release dosage form, and by pulmonary inhalation. Any form of administration will work so long as the proper dosages are delivered without destroying the active ingredient.

The term “nucleoside and nucleotide reverse transcriptase inhibitors” (“NTRI” s) as used herein means nucleosides and nucleotides and analogues thereof that inhibit the activity of HIV-1 reverse transcriptase, the enzyme which catalyzes the conversion of viral genomic HIV-1 RNA into proviral HIV-1 DNA.

Typical suitable NRTIs include zidovudine (AZT) available under the RETROVIR tradename from Glaxo-Wellcome Inc., Research Triangle, N.C. 27709; didanosine (ddI) available under the VIDEX tradename from Bristol-Myers Squibb Co., Princeton, N.J. 08543; zalcitabine (ddC) available under the HIVID tradename from Roche Pharmaceuticals, Nutley, N.J. 07110; stavudine (d4T) available under the ZERIT trademark from Bristol-Myers Squibb Co., Princeton, N.J. 08543; lamivudine (3TC) available under the EPIVIR tradename from Glaxo-Wellcome Research Triangle, N.C. 27709; abacavir (1 592U89) disclosed in WO96/30025 and available under the ZIAGEN tradename from Glaxo-Wellcome Research Triangle, N.C. 27709; adefovir dipivoxil [bis(POM)-PMEA] available under the PREVON tradename from Gilead Sciences, Foster City, Calif. 94404; lobucavir (BMS-180194), a nucleoside reverse transcriptase inhibitor disclosed in EP-0358154 and EP-0736533 and under development by Bristol-Myers Squibb, Princeton, N.J. 08543; BCH-10652, a reverse transcriptse inhibitor (in the form of a racemic mixture of BCH-10618 and BCH-10619) under development by Biochem Pharma, Laval, Quebec H7V, 4A7, Canada; emitricitabine [(−)-FTC] licensed from Emory University under Emory Univ. U.S. Pat. No. 5,814,639 and under development by Triangle Pharmaceuticals, Durham, N.C. 27707; beta-L-FD4(also called beta-L-D4C and named beta-L-2′, 3′-dicleoxy-5-fluorocytidene) licensed by Yale University to Vion Pharmaceuticals, New Haven Conn. 0651 1; and DAPD, the purine nucleoside, (−)-beta-D-2,6,-diaminopurine dioxolane disclosed in EP 0656778 and licensed by Emory University and the University of Georgia to Triangle Pharmaceuticals, Durham, N.C. 27707; and lodenosine (FddA), 9-(2,3-dideoxy-2-fluoro-b-D-threo-pentofuranosyl)adenine, a acid stable purine-based reverse transcriptase inhibitor discovered by the NIH and under development by U.S. Bioscience Inc., West Conshohoken, Pa. 19428.

The term “non-nucleoside reverse transcriptase inhibitors” (“NNRTI”s) as used herein means non-nucleosides that inhibit the activity of HIV-1 reverse transcriptase.

Typical suitable non-nucleoside reverse transcriptase inhibitors include nevirapine (BI-RG-587) available under the VIRAMUNE tradename from Boehringer Ingelheim, the manufacturer for Roxane Laboratories, Columbus, Ohio 43216; delaviradine (BHAP, U-90152) available under the RESCRIPTOR tradename from Pharmacia & Upjohn Co., Bridgewater N.J. 08807; efavirenz (DMP-266) a benzoxazin-2-one disclosed in WO94/03440 and available under the SUSTIVA tradename from DuPont Pharmaceutical Co., Wilmington, Del. 19880-0723; PNU-142721, a furopyridine-thiopyrimide underdevelopment by Pharmacia and Upjohn, Bridgewater N.J. 08807; AG-1549 (formerly Shionogi # S-1153); 5-(3,5-dichlorophenyl)-thio-4-isopropyl-1-(4-pyridyl)methyl-1H-imidazol-2-ylmethyl carbonate disclosed in WO 96 /10019 and under clinical development by Agouron Pharmaceuticals, Inc., LaJolla Calif. 92037-1020; MKC-442 1-(ethoxymethyl)-5-(1-methylethyl)-6-(phenylmethyl)-(2,4(1H,3H)-pyrimidinedione discovered by Mitsubishi Chemical Co. and under development by Triangle Pharmaceuticals, Durham, N.C. 27707; and (+)-calanolide A (NSC-675451) and B coumarin derivatives disclosed in NIH U.S. Pat. No. 5,489,697, licensed to Med Chem Research, which is co-developing (+) calanolide A with Vita-Invest as an orally administrable product.

The term “protease inhibitor” (“PI”) as used herein means inhibitors of the HIV-1 protease, an enzyme required for the proteolytic cleavage of viral polyprotein precursors (e.g., viral GAG and GAG Pol polyproteins), into the individual functional proteins found in infectious HIV-1. HIV protease inhibitors include compounds having a peptidomimetic structure, high molecular weight (7600 daltons) and substantial peptide character, e.g. CRIXIVAN(available from Merck) as well as nonpeptide protease inhibitors e.g., VIRACEPT (available from Agouron).

Typical suitable protease inhibitors include saquinavir (Ro 31-8959) available in hard gel capsules under the INVIRASE tradename and as soft gel capsules under the FORTOUASE tradename from Roche Pharmaceuticals, Nutley, N.J. 07110-1199; ritonavir (ABT-538) available under the NORVIR tradename from Abbott Laboratories, Abbott Park, Ill. 60064; indinavir (MK-639) available under the CRIXIVAN tradename from Merck & Co., Inc., West Point, Pa. 19486-0004; nelfnavir (AG-1343) available under the VIRACEPT tradename from Agouron Pharmaceuticals, Inc., LaJolla, Calif. 92037-1020; amprenavir (141W94), a non-peptide protease inhibitor under development by Vertex Pharmaceuticals, Inc., Cambridge, Mass. 02139-4211 and available from Glaxo-Wellcome, Research Triangle, N.C. under an expanded access program; lasinavir (BMS-234475) available from Bristol-Myers Squibb, Princeton, N.J. 08543 (originally discovered by Novartis, Basel, Switzerland (CGP-61755); DMP-450, a cyclic urea discovered by Dupont and under development by Triangle Pharmaceuticals; BMS-2322623, an azapeptide under development by Bristol-Myers Squibb, Princeton, N.J. 08543 as a 2nd-generation HIV-1 PI; and ABT-378 under development by Abbott , Abbott Park, Ill. 60064; and AG-1549 an orally active imidazole carbamate discovered by Shionogi (Shionogi #S-1153) and under development by Agouron Pharmaceuticals, Inc., LaJolla Calif. 92037-1020;

The term “anti-HIV-1 therapy” as used herein means any anti-HIV-1 drug found useful for treating HIV-1 infections in man alone, or as part of multidrug combination therapies, especially the triple and quadruple combination therapies called HMRT.

Typical suitable anti-HIV-1 therapies include, but are not limited to multidrug combination therapies such as (i) at least three anti-HIV-1 drugs selected from two NRTIs, one PI, a second PI, and one NNRTI; and (ii) at least two anti-HIV-1 drugs selected from, NNRTIs and PIs ;see Tables I, II and III, hereinafter.

Typical suitable HAART—multidrug combination therapies-include (a) triple combination therapies such as two NRTIs and one PI; or (b) two NRTIs and one NNRTI; and (c) quadruple combination therapies such as two NRTIs, one PI and a second PI or one NNRTI. In treatment-naive patients, it is preferred to start anti-HIV-1 treatment with the triple combination therapy; the use of two NRTIs and one PI is prefered unless there is intolerance to PIs. Drug compliance is essential. The CD4⁺ and HIV-1-RNA plasma levels should be monitored every 3-6 months. Should viral load plateau, a fourth drug,e.g., one PI or one NNRTI could be added. See the Table A hereinbelow.

TABLE A
ANTI-HIV-1 MULTI DRUG COMBINATION THERAPIES

| A. Triple Combination Therapies | |
|---|---|
| 1. | Two NRTIs¹ + one PI² |
| 2. | Two NRTIs¹ + one NNRTI1³ |

B. Quadruple Combination Therapies⁴

Two NRTIs + one PI + a second PI or one NNRTI

C. ALTERNATIVES:⁵

Two NTRI¹
One NTRI⁵ + one PI²
Two PIs⁶ ± one NTRI⁷ or NNRTI³
One PI² + one NRTI⁷ + one NNRTI³
FOOTNOTES TO TABLE A
¹One of the following: zidovudine + lamivudine; zidovudine + didanosine; stavudine + lamivudine; stavudine + didanosine; zidovudine + zalcitabine; See also Table I
²Indinavir, nelfinavir, ritonavir or saquinavir soft gel capsules. Ritonavir is used less frequently because of troublesome adverse effects. The old formulation of saquinavir was used least often because of its poor bioavailability and limited effectiveness, but the new saquinavir formulation should be more effective. See also Table III.
³Nevirapine or delavirdine. See also Table II
⁴See A-M. Vandamne et al Antiviral Chemistry + Chemotherapy 9:187 at p 193-197 and FIGS. 1 + 2.
⁵Alternative regimens are for patients unable to take a recommended regimen because of compliance problems or toxicity, and for those who fail or relapse on a recommended regimen. Double nucleoside combinations may lead to HIV- resistance and clinical failure in many patients.
⁶Most data obtained with saquinavir and ritonavir (each 400 mg bid). See also Table III
⁷Zidovudine, stavudine or didanosine. See also Table I

Other anti-HIV-1 drugs useful for administration in association with pegylated interferon alfa include hydroxyurea, ribavirin, IL-2 and IL-12, and Yissum Project No. 11607 . These above-listed anti-HIV-1 drugs may also be administered in association with pegylated interferon alfa in association with any anti-HIV-1 drug therapy, especially the triple and quadruple drug combinations called HAART.

Hydroyurea (Droxia) is a ribonucleoside triphosphate reductase inhibitor, the enzyme involved in the activation of T-cells. Hydroxyurea discovered at the NCI is under development by Bristol-Myers Squibb. In preclinical studies, it was shown to have a synergistic effect on the activity of didanosine and has been studied with stavudine.

Yissum Project No. 11607, a synthetic protein based on the HIV-1 Vif protein under preclinical development by Yissum Research Development Co., Jerusalem 91042, Israel.

The pegylated inteferon alfa, PEG₁₂₀₀₀-IFN-alfa2b(available from Schering-Plough Research Institute, Kenilworth, N.J.) increased the in vitro anti HIV-1 activity of ribavirin. The combination of PEG₁₂₀₀₀-IFN-alfa2b and ribavirin inhibited HIV replication in vitro using phytohemagylutinin (“PHA” -P)—activated peripheral blood mononuclear cells (“PBMCs”) at doses corresponding to plasmatic concentrations observed in animals and man. Healthy PBMCs were separated from a buffy-coat of one HIV-seronegative blood donor by Ficoll-Hypaque density gradient centrifugation. PBMCs were activated by 1 μg/ml phytohemagglutinin (PHA-P) for two days in cell culture medium A: RPMI 1640 supplemented with 10% heat-inactivated (+56° C., 45 min.) fetal calf plasma (FCS), 2 mM L-glutamine and a tri-antibiotic mixture (penicillin, streptomycin, neomycin; PSN). After these two days, cells were washed and cultured at one million cell per milliliter in cell culture medium B: cell culture medium A supplemented with 20 IU/ml recombinant human interleukin-2. Cells were maintained at +37° C. in a 5% CO₂-air humidified atmosphere. Experiments were repeated twice with cells of other blood donors. In total, three independent experiments were performed.

PBMCs were infected with 1,000 50% Tissue Culture Infectious Doses (TCID50) of the reference HIV-1-LAI strain [F.Barré-Sinoussi, Science, 1983, 220, 868-871]. This strain has been amplified using PHA-P-activated umbilical blood mononuclear cells (UBMC). Viral stock has been then titrated on PHA-P activated PBMC by end-point dilution. TCID50 was then calculated using Karber's formula [Arch. Exp. Path. Pharmak., 1931, 162, 126-133].

PEG₁₂₀₀₀-IFN-α2b and ribavirin, alone and in combination, and AZT used as a control, were administrated 24 hours before HIV-1 infection and maintained all along the culture. Three doses of PEG₁₂₀₀₀-IFN-α2b and ribavirin were used.

200,000 PHA-P-activated PBMCs were added to each well of 96-well microplates. Cells were 24 hour-pretreated prior to infection with the reference HIV-1-LAI strain. Twice a week, cell supernatants were collected, and drugs and medium were renewed. At day 7, RT activity were determined in cell supernatants, and potential cytotoxic effects of drugs and drug combinations were evaluated by microscopic observation.

Viral replication was measured by determining reverse transcriptase (“RT”) activity in cell supernatants using Retro-Sys® kit, according to manufacturer's recommendations (Innovagen, Lund, Sweden).

Effective doses were calculated using cumulative RT activities with Chou J. and TC. microcomputer software.

The combined effects were analyzed using either the combination index (Cl) [Chou & Talalay, 1984] with J and TC Chou microcomputer software, or the fractionary inhibitory concentration (FIC) index [Antimicrob. Agents. Chemother., 1987, 31, 1613-1617]. When the Cl or FIC index is equal to 1, the combination is additive. When it is below 1.0, the combination is synergistic, and when it is above 1.0, the combination is judged as antagonistic.

PEG₁₂₀₀₀-IFN-alfa2b as well as the combination of PEG₁₂₀₀₀-IFN-alfa2b and ribavirin inhibited the HIV replication at doses corresponding to plasmatic concentrations measured in mice and HIV-1 infected patients [BE. Gilbert, et al. Antimicrob. Agents Chemother., 1988, 32. 117-121; E. Connor at al., Antimicrob. Agents Chemother., 1993, 37, 537-539].

IL-2 is disclosed in Ajinomoto EP-0142268 , Takeda EP-0176299, and Chiron U.S. Pat. Nos. RE 33,653, 4,530,787, 4,569,790, 4,604,377, 4,748,234, 4,752,585, and 4,949,314 is available under the PROLEUKIN(aldesleukin) tradename from Chiron Corp., Emeryville, Calif. 94608-2997 as a lyophilized powder for IV infusion or sc administration upon reconstitution and dilution with water; doses of about 1 to about 20 million IU/day, sc is preferred; a dose of about 15 million IU/day, sc is more preferred.

IL-12 is disclosed in WO96/25171 and is available from Roche Pharmaceuticals, Nutley, N.J. 07110-1199 and American Home Prodocts, Madison, N.J. 07940; a dose of about 0.5 microgram/kg/day to about 10 microgram/kg/day, sc.

Pentafuside (DP-178, T-20) a 36-amino acid synthetic peptide,disclosed in U.S. Pat. No.5,464,933 licensed from Duke University to Trimeris which is developing pentafuside in collaboration with Duke University; pentafuside acts by inhibiting fusion of HIV-1 to target membranes. Pentafuside (3-100 mg/day) is given as a continuous sc infusion or injection together with efavirenz and 2 PI's to HIV-1 positive patients refractory to a triple combination therapy; use of 100 mg/day is preferred.

The term “interferon-alfa” as used herein means the family of highly homologous species-specific proteins that inhibit viral replication and cellular proliferation and modulate immune response. Typical suitable interferon-alfas include, but are not limited to, recombinant interferon alfa-2b such as Intron-A interferon available from Schering Corporation, Kenilworth, N.J., recombinant interferon alfa-2a such as Roferon interferon available from Hoffmann-La Roche, Nutley, N.J., recombinant interferon alpha-2C such as Berofor alpha 2 interferon available from Boehringer Ingelheim Pharmaceutical, Inc., Ridgefield, Conn., interferon alpha-n1, a purified blend of natural alfa interferons such as Sumiferon available from Sumitomo, Japan or as Wellferon interferon alpha-n1 (INS) available from the Glaxo-Wellcome Ltd., London, Great Britain, or a consensus alpha interferon such as those described in U.S. Pat. Nos. 4,897,471 and 4,695,623 (especially Examples 7, 8 or 9 thereof) and the specific product available from Amgen, Inc., Newbury Park, Calif., or interferon alfa-n3 a mixture of natural alfa interferons made by Interferon Sciences and available from the Purdue Frederick Co., Norwalk, Conn., under the Alferon Tradename, as well as pegylated interferon alfa, as defined herein below.The use of interferon alfa-2a or alpha 2b is preferred. Since interferon alpha 2b, among all interferons, has the broadest approval throughout the world for treating chronic hepatitis C infection, it is most preferred. The manufacture of interferon alpha 2b is described in U.S. Pat. No. 4,530,901. The use of pegylated interferon alfa-2a or pegylated interferon alpha 2b is more preferred.

The term “pegylated interferon alfa” as used herein means polyethylene glycol modified conjugates of interferon alfa, preferably interferon alfa-2a and -2b. The preferred polyethylene-glycol-interferon alfa -2b conjugate is PEG₁₂₀₀₀-interferon alfa 2b. The phrases “12,000 molecular weight polyethylene glycol conjugated interferon alpha” and “PEG₁₂₀₀₀-IFN alfa” as used herein mean conjugates such as are prepared according to the methods of International Application No. WO95/13090 and containing urethane linkages between the interferon alfa-2a or -2b amino groups and polyethylene glycol having an average molecular weight of 12000.

The preferred PEG₁₂₀₀₀-interferon alfa-2b is prepared by attaching a PEG polymer to the epsilon amino group of a lysine residue in the IFN alfa-2b molecule. A single PEG₁₂₀₀₀ molecule is conjugated to free amino groups on an IFN alfa-2b molecule via a urethane linkage. This conjugate is characterized by the molecular weight of PEG₁₂₀₀₀ attached. The PEG12000-IFN alfa-2b conjugate is formulated as a lyophilized powder for injection. The objective of conjugation of IFN alfa with PEG is to improve the delivery of the protein by significantly prolonging its plasma half-life, and thereby provide protracted activity of IFN alfa.

Other interferon alfa conjugates can be prepared by coupling an interferon alfa to a water-soluble polymer. A non-limiting list of such polymers include other polyalkylene oxide homopolymers such as polypropylene glycols, polyoxyethylenated polyols, copolymers thereof and block copolymers thereof. As an alternative to polyalkylene oxide-based polymers, effectively non-antigenic materials such as dextran, polyvinylpyrrolidones, polyacrylamides, polyvinyl alcohols, carbohydrate-based polymers and the like can be used. Such interferon alfa-polymer conjugates are described in U.S. Pat. No. 4,766,106, U.S. Pat. No. 4,917,888, European Patent Application No. 0 236 987, European Patent Application Nos. 0510 356, 0 593 868 and 0 809 996 (pegylated interferon alfa-2a) and International Publication No. WO 95/13090.

Pharmaceutical composition of pegylated interferon alfa-suitable for parenteral administration may be formulated with a suitable buffer, e.g., Tris-HCl, acetate or phosphate such as dibasic sodium phosphate/monobasic sodium phosphate buffer, and pharmaceutically acceptable excipients ( e.g., sucrose), carriers (e.g. human plasma albumin), toxicity agents (e.g. NaCl), preservatives (e.g. thimerosol, cresol or benylalcohol), and surfactants( e.g. tween or polysorabates) in sterile water for injection. The pegylated interferon alfa-may be stored as lyophilized powders under a refrigeration at 2°-8° C. The reconstituted aqueous solutions are stable when stored between 2° and 8° C. and used within 24 hours of reconstitution. See for example U.S. Pat. Nos, 4,492,537; 5,762,923 and 5,766,582. The reconstituted aqueous solutions may also be stored in prefilled, multi-dose syringes such as those useful for delivery of drugs such as insulin. Typical suitable syringes include systems comprising a prefilled vial attached to a pen-type syringe such as the NOVOLET Novo Pen available from Novo Nordisk, as well as prefilled, pen-type syringes which allow easy self-injection by the user. Other syringe systems include a pen-type syringe comprising a glass cartridge containing a diluent and lyophilized pegylated interferon alfa powder in a separate compartment.

A person suffering from chronic hepatitis C infection may exhibit one or more of the following signs or symptoms:

(a) elevated ALT,

(b) positive test for anti-HCV antibodies,

(c) presence of HCV as demonstrated by a positive test for the presence of HCV-RNA in the serum,

(d) clinical stigmata of chronic liver disease,

(e) hepatocelluar damage.

To practice the invention, the combination therapy of pegylated interferon-alfa and ribavirin is administered in association with anti-retroviral therapy,e.g., HAART, to the patient having HIV-1 infection and exhibiting one or more of the above signs or symptoms in the first and second treatment time periods in amounts sufficient to eliminate or at least alleviate one or more of the signs or symptoms., and to lower the HCV-RNA serum levels by at least a power of ten, and preferably to eradicate detectable HCV-RNA at least by the end of the second treatment time period and to maintain no detectable HCV-RNA for at least 24 weeks after the end of the second treatment time period. The sum of the first and second treatment time periods is about 40-50 weeks, and preferably is 48 weeks. Administration of the ribavirin may be discontinued after the end of the second time period depending upon the judgment of the attending clinician.

The term “no detectable HCV-RNA” in the context of the present invention means that there are fewer than 100 copies of HCV-RNA per ml of serum of the patient as measured by quantitative, multi-cycle reverse transcriptase PCR methodology. HCV-RNA is preferably measured in the present invention by research-based RT-PCR methodology well known to the skilled clinician. This methodology is referred to herein as HCV-RNA/qPCR. The lower limit of detection of HCV-RNA is 100 copies/mL. Serum HCV-RNA/qPCR testing and HCV genotype testing will be performed by a central laboratory. See also J. G. McHutchinson et al. (N. Engl. J. Med., 1998, 339:1485-1492), and G. L. Davis et al. (N. Engl. J. Med. 339:1493-1499).

In a preferred embodiment of the present invention, those patients co-infected with HIV-1 and HCV infections are treated with pegylated interferon alfa in combination with ribavirin and a HAART combination considered appropriate by the attending clinician and the patient; use of the interferon alfa-2b-ribavirin combination therapy sold by Schering Corp. under the REBETRON tradename is preferred. See also J. G. McHutchinson et al. (N. Engl. J. Med., 1998, 339:1485-1492), and G. L. Davis et al. (N. Engl. J. Med. 339:1493-1499). Ribavirin, 1-β-D-ribofuranosyl-1 H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771.

For the pediatric patient co-infected with the HIV-1 and HCV infections, a suitable HAART includes a NRTI+a PI, e.g., Nelfinavir +a NNRTI, e.g., Efavirenz in combination with the dosages and dosage regimens for pegylated interferon alfa and ribavirin listed herein above. See also Tables I-IV herein below. A human growth hormone such as the polypeptide hormone, somatropin, of recombinant rDNA origin, available under the HUMATROPE tradename from Eli Lilly & Co., Indianapolis, Ind. 46285, may be administered to these pediatric patients in the dosage and administration schedule listed in the product information sheet in consultation with the attending clinician to reduce retardation of growth associated with pegylated interferon alfa treatment.

HAART is administered to the patient in association with pegylated interferon-alfa, that is, the pegylated interferon-alfa dose may be administered before, after or during the same period of time that the patient receives doses of HAART. A human growth hormone such as the polypeptide hormone, somatropin, of recombinant rDNA origin, available under the HUMATROPE tradename from Eli Lilly & Co., Indianapolis, Ind. 46285, may also be administered—in association with HAART and pegylated interferon alfa—to the pediatric patient having HIV-1 infection in the dosage and administration schedule listed in the product information sheet in consultation with the attending clinician.

In a preferred embodiment of the present invention, pegylated interferon alfa is administered to HIV-1 infected patients prior to initiation of HAART, and preferably about two to about four weeks prior to initiation of HAART. In another preferred embodiment of the present invention, administeration of pegylated interferon alfa is initiated concurrently, i.e., on the same day with the administeration of HAART. In another preferred embodiment of the present invention the pegylated interferon-alfa is administered after the HIV-1 infected patient has initiated HAART.

The goal of the anti-HIV-1 therapy of the present invention is to reduce the HIV-1-RNA viral load below the detectable limit. The “detectable limit of HIV-1-RNA” in the context of the present invention means that there are fewer than about 200 to fewer than about 50 copies of HIV-1-RNA per ml of plasma of the patient as measured by quantitative, multi-cycle reverse transcriptase PCR methodology. HIV-1-RNA is preferably measured in the present invention by the methodology of Amplicor-1 Monitor 1.5 (available from Roche Diagnsotics)or of Nuclisens HIV-1 QT-1. This methodology is described by Schooley, RT, Antiviral Therapy(1997), 2 (Suppl. 4):59-70.

The doses and dosage regimen of the NRTIs, NNRTIs and PI; IL-2, IL-12 and pentafuside will be determined by attending clinician in view of the approved doses and dosage regimen in the package insert or as set forth in the protocol taking into consideration the age, sex and condition of the patient and the severity of the HIV-1 infection. For the pediatric patient infected with the HIV-1, or co-infected with the H IV-1 and HCV infections a suitable HAART includes a NRTI+a PI, e.g., Nelfinavir +a NNRTI, e.g., Efavirenz in combination with the dosages and dosage regimens for pegylated interferon alfa and ribavirin listed herein above. See also Tables I-IV hereinafter for dosages and dosage regimens.

The following clinical protocols may be used to administer the anti-HIV-1 therapy of the present invention. Many modifications of this clinical protocol will be obvious to the skilled clinician, and the following Study Designs should not be interpreted as limiting the scope of the method of this invention which is only limited by the claims listed hereinafter. See for example J. G. McHutchinson et al. (N. Engl. J. Med., 1998, 339:1485-1492), and G. L. Davis et al. (N. Engl. J. Med. 339:1493-1499).

### Study No. 1

The study population will include male and female patients diagnosed with HIV-1 infection who are either treatment naive or treatment-experienced and will be included if they meet the following inclusion and exclusion criteria:

### Subject Inclusion Criteria

Subjects diagnosed with HIV-1 infection who are either treatment naive or treatment-experienced.

HIV-RNA by Amplicor test, Version 1.5 of greater than 500 copies/mL.

CD₄⁺ count greater than 100 copies/ml, preferably greater than 200 cells/mL.

Subjects in good physical health with clinically acceptable safety laboratory test results and ECG.

The following laboratory parameters must be met:

Platelet count≧75,00/mL

Hemoglobin 9 gm/dL (90 gm/L)

Absolute neutrophil count 1500/μL

Creatinine∠5 times the upper limit of normal

SGOT/SGPT≦5×upper limit of normal

Bilirubin≦2.5×upper limit of normal

A negative urine pregnancy test (females only)

Subjects must be willing and able to give written informed consent and be able to adhere to the schedule set forth in the protocol.

### Subject Exclusion Criteria

Females who are breast-feeding or pregnant or who are not using adequate birth control.

Subject with allergy to E. coli proteins

Subjects with a significant past medical/psychiatric history, specifically depression or dementia.

The subjects will be randomized to receive pegylated interferon alfa 2b, i.e., PEG₁₂₀₀₀-interferon alfa 2b at doses between 0.5 and 4.5 micrograms per kilogram e.g. at doses of 0.5, 1.0, 1.5, 3.0 or 4.5 micrograms per kilogram by subcutaneous injection once a week. HAART may also be initiated before or concurrently with the administration of the pegylated interferon alfa 2b, i.e., PEG₁₂₀₀₀-interferon alfa 2b,i.e., PEG-Intron which is available from Schering Corp, Kenilworth, N.J.

### Overall Design and Plan of the Study

The primary efficacy objective will be lowering of the HIV-1-RNA plasma levels to below the limit of quantitation.(LOQ), i.e., less than 50 copies of HIV-RNA per mL of plasma.

Plasma HIV-1-RNA/qPCR testing will be performed by a central laboratory. After a sufficient time below the limit of quantitation (preferably greater than one year) all anti-retroviral therapy will be discontinued until viral rebound occurs to a level ≧10,000 copies of HIV-RNA per mL of plasma at which time an additional course of anti-retroviral therapy will be initiated. After a sufficient time below the LOQ, an additional STI wherein interferon-alfa, preferably pegylated interferon-alfais administered in accordance with the present invention will be initiated. The cycle of treatment followed by treatment interruption will be repeated until viral rebound during STI remain below 10,000 copies of HIV-RNA per mL of plasma,preferably below 5,000 copies of HIV-RNA per mL of plasma, in the absence of any anti-retroviral therapy or nterferon-alfa.

### Study No. 2

### Study Objectives

The study population will include male and female patients diagnosed with HIV-1 infection who have maintained viral suppression below the limit of quantitation for a sufficient length of time (preferably greater than one year).

Subject Inclusion Criteria

Subjects diagnosed with HIV-1 infection who are either treatment naive or treatment-experienced.

HIV-RNA by Amplicor test, Version 1.5 of less than 50 copies/mL.

CD₄⁺ count greater than 100 copies/ml, preferably greater than 200 cells/mL.

Subjects in good physical health with clinically acceptable safety laboratory test results and ECG.

The following laboratory parameters must be met:

Platelet count≧75,00/mL

Hemoglobin 9 gm/dL (90 gm/L)

Absolute neutrophil count 1500/μL

Creatinine∠1.5 times the upper limit of normal

SGOT/SGPT≦5×upper limit of normal

Bilirubin≦2.5×upper limit of normal

A negative urine pregnancy test (females only) Subjects must be willing and able to give written informed consent and be able to adhere to the schedule set forth in the protocol.

HIV RNA less than 5° copies/mL

### Subject Exclusion Criteria

Females who are breast-feeding or pregnant or who are not using adequate birth control.

Subject with allergy to E. coli proteins

Subjects with a significant past medical/psychiatric history, specifically depression or dementia.

### Overall Design and Flow of the Study

Subjects will be randomized to receive pegylated interferon alfa or nothing during a STI. The time to viral rebound and percentage of patients requiring reinitiation of HAART for viral RNA levels≧10,000 copies/mL will be the primary endpoint. Those patients requiring re-initiation of HAART will remain allocated to either the pegylated interferon-alfa or nothing arm for subsequent cycles (preferably 6 months up to one year in duration). The primary endpoints during each cycle of STI will be time to viral rebound and magnitude of rebound.

TABLE I
NUCLEOSIDE REVERSE TRANSCRIPTASE INHIBITORS (NRTI)
DOSAGE & DOSAGE REGIMEN

| NRTI (Tradename, Marketer) | Usual adult dosage |
|---|---|
| Zidovudine, AZT (Retrovir - Glaxo | 200 mg PO tid or |
| Wellcome)* | 300 mg PO bid |
| Stavudine (Zerit - Bristol-Myers Squibb)* | 40 mg PO bid¹ |
| Didanosine (Videx - Bristol-Myers Squibb)* | 200 mg PO bid² |
| Lamivudine (Epivir - Glaxo Wellcome)* | 150 mg PO bid³ |
| Zalcitabine (Hivid - Roche) | 0.75 mg PO tid |
| Zidovudine plus lamivudine (Combivir- | 1 tablet PO bid⁴ |
| Glaxo Wellcome) | |
| Abacavir (Ziagen-Glaxo-Wellcome) | 200 or 400 mg PO tid |
| Adefovir dipivoxil (Prevon-Gilead Sciences) | 125 or 200 mg PO qd⁵ |
| Lobucavir (BMS-180194-BMS) | 200 mg PO bid⁶ |
| BCH-10652 (Biochem Pharma) | 400 mg PO, qid⁷ |
| Emitricitabine ((−)-FTC-Triangle | 200 mg PO qd⁸ |
| Pharmaceuticals) | |
| Beta-L-FD4 (B-L-D4C-Vion Pharmaceutical) | 0.2-25 mg/ky/day⁹ |
| DAPD (Triangle Pharmaceuticals) | —¹⁰ |
| Lodenosine (FddA-U.S. Bioscience) | 1.6-3.2 mg/Kg PO bid¹¹ |

Footnotes Table I
*Available in a liquid formulation.
¹For patients less than 60 kg, 30 mg PO bid.
²With tablets; for patients <60 kg. 125 mg PO bid; >60 kg. 200 mg PO bid;. With powder, dosage varies from 167 mg (<60 kg) to 250 mg PO (<60 kg) bid. Doses should be taken at least 30 minutes before meals or at least two hours afterward.
³For patients less than 50 kg. 2 mg/kg PO bid.
⁴Each tablet contains 300 mg of zidovudine and 150 mg of lamivudine.
⁵Available under an expanded access program - a NIH-sponsored Phase III Trial
⁶Phase II
⁷Phase I/II; see PharmaProjects, sections J5A & J5Z
⁸Phase II/III; see PharmaProjects, sections J5A & J5Z
⁹Preclinical; active in duck HBV model; see PharmaProjects, sections J5A & J5Z
¹⁰Preclinical; active po and IV; DAPD is a prodrug of another dioxolene purine, DXG. See PharmaProjects, sections J5A & J5Z
¹¹Phase II, FddA has potential for once-a-day dosage

TABLE II
NON-NUCLEOSIDE REVERSE TRANSCRIPTASE
INHIBITORS (NNRTI) Dosage and Dosage Regimen

| | Usual adult dosage |
|---|---|
| NRTI (Tradename, Marketer) | and Dosage Regimen |
| Nevirapine (Viramune - Roxane) | 200 mg PO bid¹ |
| Detavirdine (Rescriptor - Pharmacia & Upjohn) | 400 mg PO tid |
| Efavirenz (Sustiva, Dupont) | 200 mg PO qid² |
| PNU-142721 (Pharmacia + Upjohn) | —³ |
| AG-1549 (Agouvon Pharmaceuticals) | —⁴ |
| MKC-442 (Triangle Pharmaceuticals) | 750 mg PO bid⁵ |
| (+)-Calanolide A (Med Chem Research) | 800 mg PO⁶ |

¹For the first two weeks of treatment with nevirapine, to decrease the risk of rash, patients should take only one 200-mg tablet per day.
²Quadruple Therapy of efavirenz with indinavir + 2 NRTIs or Triple Therapy of efavirenz + AZT + lamivudine.
³Preclinical Phase; see PharmaProjects, sections J5A & J5Z
⁴Phase I/II evaluating dose and comcomitant use with other anti-HIV-1 therapies; see Pharmaprojects, sections J5A & J5Z.
⁵Triple Therapy of (a) MKC-442 with stavudine and either lamivudine or didanosine or (b) MKC-442 with nelfinavir (qv) and NRTIs.
⁶Phase I; see PharmaProjects, sections J5A & J5Z

TABLE III
Protease Inhibitor (PI) Dosage and Dosage Regimen

| PI (Tradename, Marketer) | Dosage + Dosage Regimen |
|---|---|
| Saquinavir (Invirase - hard gel | 600 mg PO tid¹ |
| capsule-Roche) (Fortovase - soft gel | 1100 mg PO tid¹ |
| capsule-Roche) | |
| Ritonavir (Norvir - Abbott) | 600 mg PO bid² |
| Indinavir (Crixivan - Merck) | 800 mg PO qid³ |
| Nelfinavir (Viracept - Agouron) | 750 mg PO tid⁴ |
| Amprenavir (141W94, Glaxo) | 900 mg-1200 mg PO bid⁵ |
| Lasinavir (BMS-234475, BMS) | —⁶ |
| DMP-450 (Triangle Pharmaceuticals) | —⁷ |
| BMS-2322623 (BMS) | —⁸ |
| ABT-378 (Abbott) | 60 mg PO bid⁹ |

¹With, or within two hours after, a full meal.
²With food. The liquid formulation has an unpleasant taste; the manufacturer suggests taking it with chocolate milk or a liquid nutritional supplement.
³With water, one hour before or two hours after a meal. Patients taking indinavir should drink at least 48 ounces (1.5 liter) of water daily.
⁴With food.
⁵Quadruple Combination Therapy of amprenavir with AZT + lamivudine + abacavir.
⁶Phase I/II; see Pharmaprojects, sections J5A & J5Z.
⁷Phase II; see Pharmaprojects, sections J5A & J5Z.
⁸Preclinical Studies; Prodrug esters of BMS 2322623 enhance oral absorption; see Pharmaprojects, sections J5A & J5Z.
⁹Phase I Studies show ABT-378 to be 10X more potent than ritonavir; see PharmaProjects sections J5A & J5Z.

TABLE IV
Other Anti-HIV-1 Drugs

| | Usual Adult Dosage |
|---|---|
| Drug (Trade Name, Marketer) | and Dosage Regimen |
| Hydroxyurea (Droxia, BMS) | 1000 mg PO qid¹ |
| Ribavirin (Rebetol, Schering-Plough) | 600 mg-1200 mg/day, PO |
| IL-2 (Proleukin, Chiron Corp.) | 1-20 milliom IU/day, sc |
| IL-12 (Roche) | 0.5-10 micrograms/kg/day, sc |
| Yissum Project No. 11607 (Yissum) | —² |

¹Triple Therapy of hydroxyurea with 400 mg ddl + 500 mg AZT; see PharmaProjects, section B3C1
²Preclinical; see Pharmaprojects, sections J5A & J5Z.

We claim:

1. A method of promoting an HIV-1 specific immune response in a patient having an HIV-1 infection in need of such promoting which comprises administering to such patients an effective amount of interferon alfa.

2. The method of claim 1 wherein the patient is a treatment-experienced patient.

3. The method of claim 1 wherein the patient is a treatment-experienced patient who has discontinued an anti-HIV-therapy.

4. The method of claim 1 wherein the patient is a treatment-experienced patient who has discontinued HAART.

5. The method of claim 1 wherein the patient is a treatment-naive patient.

6. The method of claim 1, wherein the interferon-alfa administered is interferon alfa-2a , interferon alfa-2b, pegylated interferon alfa-2a or pegylated interferon alfa-2b.

7. A method of promoting an HIV-1 specific immune response to in a patient having an HIV-1 infection in need of such promoting which comprises administering to such patients an effective amount of pegylated interferon alfa.

8. The method of claim 7 wherein the patient is a treatment-experienced patient.

9. The method of claim 7 wherein the patient is a treatment-experienced patient who has discontinued an anti-HIV-therapy.

10. The method of claim 7 wherein the patient is a treatment-experienced patient who has discontinued HAART.

11. The method of claim 7 wherein the patient is a treatment-naive patient.

12. The method of claim 7, wherein the pegylated interferon-alfa administered is pegylated interferon alfa-2a or pegylated interferon alfa-2b.

13. The method of claim 7, wherein the pegylated interferon-alfa administered is pegylated interferon alfa-2b and the effective amount is in the range of about 0.5 to about 3.0 micrograms per kilogram of pegylated interferon-alfa-2b once a week.

14. The method of claim 7, wherein the pegylated interferon-alfa administered is pegylated interferon alfa-2b and the effective amount is in the range of about 0.75 to about 1.5 micrograms per kilogram of pegylated interferon-alfa-2b administered once a week.

15. The method of claim 7, wherein the pegylated interferon-alfa administered is pegylated interferon alfa-2b and the effective amount is in the range of about 1.5 micrograms per kilogram of pegylated interferon-alfa-2b administered once a week.

16. The method of claim 7, wherein the pegylated interferon-alfa administered is a pegylated interferon alfa-2a and the effective amount of pegylated interferon alfa-2a administered is in the range of about 50 to about 500 micrograms per week.

17. The method of claim 7, wherein the pegylated interferon-alfa administered is a pegylated interferon alfa-2a and the effective amount of pegylated interferon alfa-2a administered is in the range of about 180 to about 250 micrograms per week.

18. A method of promoting HIV-1-specific T-cell activity in a patient having an HIV-1 infection who has discontinued anti-HIV therapy which comprises administering to such a patient an amount of interferon alpha for a time sufficient to lower HIV-RNA plasma level of the patient to a level below the patient's HIV-RNA plasma level prior to initiation of the anti-HIV-therapy.

19. The method of claim 18 wherein the anti-HIV-therapy is HAART.

20. The method of claim 18, wherein the interferon-alfa administered is interferon alfa-2a, interferon alfa-2b, pegylated interferon alfa-2a or pegylated interferon alfa-2b.

21. The method of claim 18 which further comprises re-initiating administering an effective amount of an anti-HIV therapy for a time sufficient to lower HIV-RNA plasma levels below the detectable limit.

22. The method of claim 21 which further comprises discontinuing anti-HIV therapy and administering to such a patient an amount of interferon alpha for a time sufficient to lower HIV-RNA plasma level of the patient to a level below the patient's HIV-RNA plasma level prior to initiation of the anti-HIV-therapy.

23. The method of claim 22 which further comprises re-initiating administering an effective amount of an anti-HIV therapy for a time sufficient to lower HIV-RNA plasma levels below the detectable limit(50 HIV-RNA copies per mL of plasma).

24. The method of claim 22 which further comprises discontinuing anti-HIV therapy and administering to such a patient an amount of interferon alpha for a time sufficient to lower HIV-RNA plasma level of the patient to a level below the patient's HIV-RNA plasma level prior to initiation of the anti-HIV-therapy.

25. A method of promoting HIV-1-specific T-cell activity in a patient having an HIV-1 infection who has discontinued anti-HIV therapy which comprises administering to such a patient an amount of pegylated interferon alpha for a time sufficient to lower HIV-RNA plasma level of the patient to a level below the patient's HIV-RNA plasma level prior to initiation of the anti-HIV-therapy.

26. The method of claim 25 wherein the anti-HIV-therapy is HAART.

27. The method of claim 25, wherein the pegylated interferon-alfa administered is pegylated interferon alfa-2a or pegylated interferon alfa-2b.

28. The method of claim 25 which further comprises re-initiating administering an effective amount of an anti-HIV therapy for a time sufficient to lower HIV-RNA plasma levels below the detectable limit(50 HIV-RNA copies per mL of plasma).

29. The method of claim 25 which further comprises discontinuing anti-HIV therapy and administering to such a patient an amount of pegylated interferon alpha for a time sufficient to lower HIV-RNA plasma level of the patient to a level below the patient's HIV-RNA plasma level prior to initiation of the anti-HIV-therapy.

30. The method of claim 25 which further comprises re-initiating administering an effective amount of an anti-HIV therapy for a time sufficient to lower HIV-RNA plasma levels below the detectable limit(50 HIV-RNA copies per mL of plasma).

31. The method of claim 25 which further comprises discontinuing anti-HIV therapy and administering to such a patient an amount of pegylated interferon alpha for a time sufficient to lower HIV-RNA plasma level of the patient to a level below the patient's HIV-RNA plasma level prior to initiation of the anti-HIV-therapy.

32. The method of claim 25, wherein the pegylated interferon-alfa administered is pegylated interferon alfa-2b and the effective amount is in the range of about 0.5 to about 3.0 micrograms per kilogram of pegylated interferon-alfa-2b once a week.

33. The method of claim 25, wherein the pegylated interferon-alfa administered is pegylated interferon alfa-2b and the effective amount is in the range of about 0.75 to about 1.5 micrograms per kilogram of pegylated interferon-alfa-2b administered once a week.

34. The method of claim 25, wherein the pegylated interferon-alfa administered is pegylated interferon alfa-2b and the effective amount is in the range of about 1.5 micrograms per kilogram of pegylated interferon-alfa-2b administered once a week.

35. The method of claim 25 wherein the pegylated interferon-alfa administered is a pegylated interferon alfa-2a and the effective amount of pegylated interferon alfa-2a administered is in the range of about 50 to about 500 micrograms per week.

36. The method of claim 25, wherein the pegylated interferon-alfa administered is a pegylated interferon alfa-2a and the effective amount of pegylated interferon alfa-2a administered is in the range of about 180 to about 250 micrograms per week.

37. A method of promoting HIV-1-specific T-cell activity in a patient having an HIV-1 infection which comprises administering to such a patient an effective amount of interferon alpha in association with an effective amount an anti-HIV therapy for a time sufficient to effect such promoting.

38. The method of claim 37 wherein the HIV-1-specific T-cells are cytotoxic T-lymphocytes.

39. The method of claim 37 wherein the anti-HIV-therapy is HAART.

40. The method of claim 37, wherein the interferon-alfa administered is interferon alfa-2a, interferon alfa-2b., consensus interferon-alfa, pegylated interferon alfa-2a or pegylated interferon alfa-2b.

41. A method of promoting HIV-1-specific T-cell activity in a patient having an HIV-1 infection which comprises administering to such a patient an effective amount of pegylated interferon alpha in association with an effective amount an anti-HIV therapy for a time sufficient to effect such promoting.

42. The method of claim 41 wherein the HIV-1-specific T-cells are cytotoxic T-lymphocytes.

43. The method of claim 41 wherein the anti-HIV-therapy is HAART.

44. The method of claim 43, wherein the pegylated interferon-alfa administered is pegylated interferon alfa-2a or pegylated interferon alfa-2b.

45. The method of claim 41, wherein the pegylated interferon-alfa administered is pegylated interferon alfa-2b and the effective amount is in the range of about 0.5 to about 3.0 micrograms per kilogram of pegylated interferon-alfa-2b once a week.

46. The method of claim 41, wherein the pegylated interferon-alfa administered is pegylated interferon alfa-2b and the effective amount is in the range of about 0.75 to about 1.5 micrograms per kilogram of pegylated interferon-alfa-2b administered once a week.

47. The method of claim 41, wherein the pegylated interferon-alfa administered is pegylated interferon alfa-2b and the effective amount is in the range of about 1.5 micrograms per kilogram of pegylated interferon-alfa-2b administered once a week.

48. The method of claim 41, wherein the pegylated interferon-alfa administered is a pegylated interferon alfa-2a and the effective amount of pegylated interferon alfa-2a administered is in the range of about 50 to about 500 micrograms per week.

49. The method of claim 41, wherein the pegylated interferon-alfa administered is a pegylated interferon alfa-2a and the effective amount of pegylated interferon alfa-2a administered is in the range of about 180 to about 250 micrograms per week.
